(19) United States
(12) Patent Application Publication
(10) Pub. No.: US 2018/0095296 A1
(43) Pub. Date: Apr. 5, 2018

(54) CONTACT LENS AND CONTACT LENS PRODUCT

(71) Applicant: LARGAN MEDICAL CO., LTD., Taichung City (TW)

(72) Inventors: En-Ping Lin, Taichung City (TW); Wei-Yuan CHEN, Taichung City (TW); Chun-Hung Teng, Taichung City (TW)

(21) Appl. No.: 15/498,817

(22) Filed: Apr. 27, 2017

(30) Foreign Application Priority Data
Sep. 30, 2016 (TW) 105131731

(51) Int. Cl. G02C 7/10; G02C 7/04; A61F 9/00

(52) U.S. Cl. CPC G02C 7/105; G02C 7/04; G02C 7/102; A61F 9/0017; G02C 7/049; G02C 7/041; G02C 7/108

(57) ABSTRACT

The present disclosure relates to a contact lens. The contact lens includes an optical region and at least one light-blocking annular region. The at least one light-blocking annular region surrounds a central point of the contact lens. When an average transmittance of visible light of the at least one light-blocking annular region is Tavg, a minimum diameter of the at least one light-blocking annular region is CDmin, and a maximum diameter of the contact lens is D, certain conditions relating to Tavg, CDmin, and D are satisfied.

### RELATED APPLICATIONS

This application claims priority to Taiwan Application Serial Number 105131731, filed Sep. 30, 2016, which is herein incorporated by reference.

### BACKGROUND

### Technical Field

The present disclosure relates to a contact lens and a contact lens product. More particularly, the present disclosure relates to a contact lens and a contact lens product, in which the amount of light passing through the contact lens can be controlled and the image quality can be improved.

### Description of Related Art

Eyes are the window of soul. However, due to the innate defects or improper uses, the proportion of people with abnormal vision is gradually increasing. Common vision problems include myopia, hyperopia or astigmatism. People with myopia has a cornea with a stronger refractive power, so that the image is focused before the retina. In contrary, people with hyperopia has a cornea with a weaker refractive power, so that the image is focused behind the retina. People with astigmatism has unequal radian in different regions of the cornea surface or has an uneven cornea surface, so that lights cannot be accurately focused on the retina so as to form a clear image.

People usually wear framed glasses or contact lenses for correcting the aforementioned vision problems. When wearing the framed glasses, it is inconvenient to do outdoor activities and cannot satisfy the aesthetic requirement. Accordingly, the number of contact lenses wearers is increasing.

However, the amount of light passing through the conventional contact lens cannot be well controlled. When the amount of light passing through the contact lens is excessive, it results in photophobia. When the amount of light passing through the contact lens is insufficient, the image is dark and unclear. Therefore, how to improve the structure and/or material of the contact lens to effectively control the amount of light passing therethrough, which can prevent the photophobia caused by excessive amount of light entering into eyes meanwhile can ensure the image brightness by maintaining sufficient amount of light passing therethrough, has become the goal of the relevant industry and academics.

### SUMMARY

According to one aspect of the present disclosure, a contact lens includes an optical region and at least one light-blocking annular region. The at least one light-blocking annular region surrounds a central point of the contact lens. When an average transmittance of visible light of the at least one light-blocking annular region is Tavg, a minimum diameter of the at least one light-blocking annular region is CDmin, and a maximum diameter of the contact lens is D, the following conditions are satisfied:

0%<Tavg≦60%; and

0.01≦CDmin/D≦0.45.

According to another aspect of the present disclosure, a contact lens product includes a contact lens and a soaking solution, wherein the contact lens is immersed in the soaking solution. The contact lens includes an optical region and at least one light-blocking annular region. The at least one light-blocking annular region surrounds a central point of the contact lens. At least one of the contact lens and the soaking solution includes a cycloplegic agent. When an average transmittance of visible light of the at least one light-blocking annular region is Tavg, a minimum diameter of the at least one light-blocking annular region is CDmin, and a maximum diameter of the contact lens is D, the following conditions are satisfied:

0%<Tavg≦60%; and

0.01≦CDmin/D≦0.80.

According to yet another aspect of the present disclosure, a contact lens includes an optical region and at least one light-blocking annular region. The at least one light-blocking annular region surrounds a central point of the contact lens. At least one of the optical region and the at least one light-blocking annular region includes a photochromic material. When an average transmittance of visible light of the at least one light-blocking annular region is Tavg, a minimum diameter of the at least one light-blocking annular region is CDmin, and a maximum diameter of the contact lens is D, the following conditions are satisfied:

0%<Tavg≦60%; and

0.01≦CDmin/D≦0.80.

According to further another aspect of the present disclosure, a contact lens includes an optical region and at least one light-blocking annular region.

The at least one light-blocking annular region surrounds a central point of the contact lens. When an average transmittance of visible light of the at least one light-blocking annular region is Tavg, a minimum diameter of the at least one light-blocking annular region is CDmin, a maximum diameter of the contact lens is D, and a base curve of the contact lens is BC, the following conditions are satisfied:

0%<Tavg≦60%;

0.01≦CDmin/D≦0.80; and

9.00 mm≦BC.

### BRIEF DESCRIPTION OF THE DRAWINGS

The present disclosure can be more fully understood by reading the following detailed description of the embodiment, with reference made to the accompanying drawings as follows:

FIG. 1A is a schematic view of a contact lens according to one embodiment of the present disclosure;

FIG. 1B is a side view of the contact lens in FIG. 1A;

FIG. 2 is a schematic view of a contact lens product according to another embodiment of the present disclosure;

FIG. 3 is a schematic view of a contact lens according to the 1st example of the present disclosure;

FIG. 4 is a schematic view of a contact lens according to the 2nd example of the present disclosure;

FIG. 5 is a schematic view of a contact lens according to the 3rd example of the present disclosure;

FIG. 6 is a schematic view of a contact lens according to the 4th example of the present disclosure;

FIG. 7 is a schematic view of a contact lens according to the 5th example of the present disclosure;

FIG. 8 is a schematic view of a contact lens according to the 6th example of the present disclosure;

FIG. 9 is a schematic view of a contact lens according to the 7th example of the present disclosure;

FIG. 10 is a schematic view of a contact lens according to the 8th to example of the present disclosure;

FIG. 11 is a schematic view of a contact lens according to the 9th example of the present disclosure;

FIG. 12 is a schematic view of a contact lens according to the 10th example of the present disclosure;

FIG. 13 is a schematic view of a contact lens according to the 11th example of the present disclosure;

FIG. 14 is a schematic view of a contact lens according to the 12th example of the present disclosure; and

FIG. 15 is a schematic view of a contact lens according to the 13th example of the present disclosure.

### DETAILED DESCRIPTION

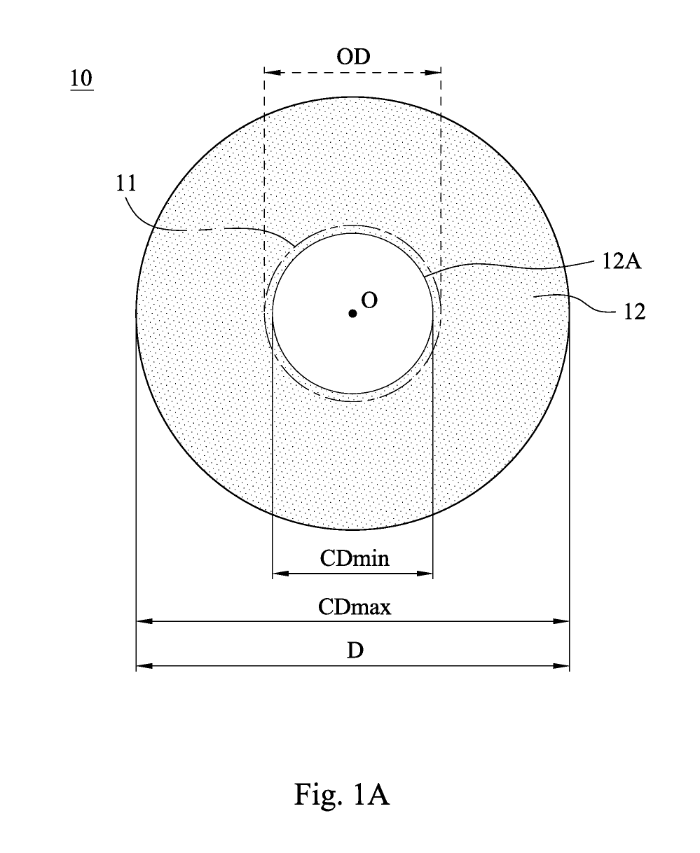

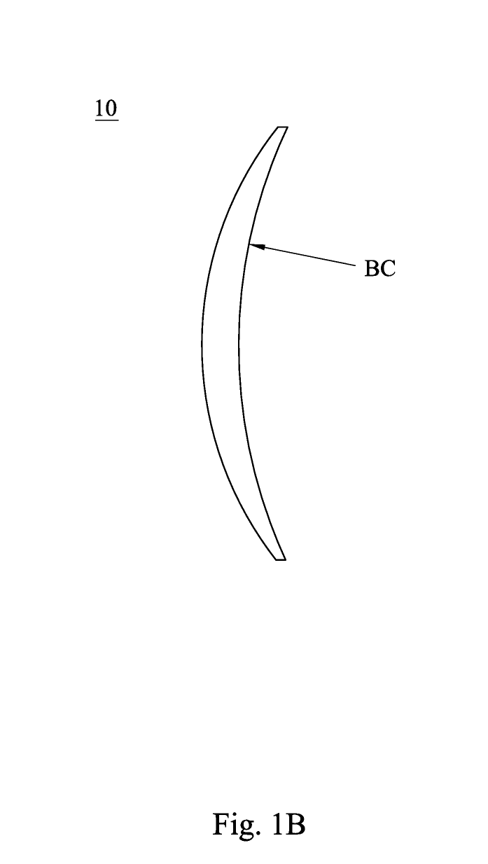

FIG. 1A is a schematic view of a contact lens 10 according to one embodiment of the present disclosure. In FIG. 1A, the contact lens 10 includes an optical region 11 and a light-blocking annular region 12. The light-blocking annular region 12 surrounds a central point O of the contact lens 10.

Specifically, the optical region 11 is the region circled by the dashed line in FIG. 1A, and the light-blocking annular region 12 is the region sprinkled with dots. In the embodiment, the optical region 11 and the light-blocking annular region 12 are partially overlapped. In other embodiment, the optical region 11 and the light-blocking annular region 12 are not overlapped. That is, the optical region 11 can be completely staggered with the light-blocking annular region 12. Moreover, the light-blocking annular region 12 and the optical to region 11 can be concentric. Alternatively, the light-blocking annular region 12 and the optical region 11 can share different center.

When an average transmittance of visible light (400 nm-700 nm) of the light-blocking annular region 12 is Tavg, the following condition is satisfied: 0%<Tavg≦60%. Therefore, an excessive amount of light passing through the is contact lens 10 can be prevented meanwhile the amount of light is sufficient, which can ease the photophobia and can eliminate the peripheral aberrations. Accordingly, the clearness and the sharpness of the image of the visual field can be enhanced. Alternatively, the following condition can be satisfied: 0%<Tavg≦50%. Alternatively, the following condition can be satisfied: 1%<Tavg≦50%. Alternatively, the following condition can be satisfied: 0%<Tavg≦40%. Alternatively, the following condition can be satisfied: 5%<Tavg≦40%. Alternatively, the following condition can be satisfied: 0%<Tavg≦30%. Alternatively, the following condition can be satisfied: 10%<Tavg≦30%. Alternatively, the following condition can be satisfied: 0%<Tavg≦20%. Alternatively, the following condition can be satisfied: 10%<Tavg≦20%. Alternatively, the following condition can be satisfied: 0%<Tavg≦10%.

When a minimum diameter of the light-blocking annular region 12 is CDmin, and a maximum diameter of the contact lens 10 is D, the following condition is satisfied: 0.01≦CDmin/D≦0.80. Therefore, an excessive amount of light passing through the contact lens 10 can be prevented meanwhile the amount of light is sufficient, which can ease the photophobia and can eliminate the peripheral aberrations. Accordingly, the clearness and the sharpness of the image of the visual field can be enhanced. Alternatively, the following to condition can be satisfied: 0.01≦CDmin/D≦0.45. Alternatively, the following condition can be satisfied: 0.05≦CDmin/D≦0.45. Alternatively, the following condition can be satisfied: 0.10≦CDmin/D≦0.40. Alternatively, the following condition can be satisfied: 0.20≦CDmin/D≦0.40.

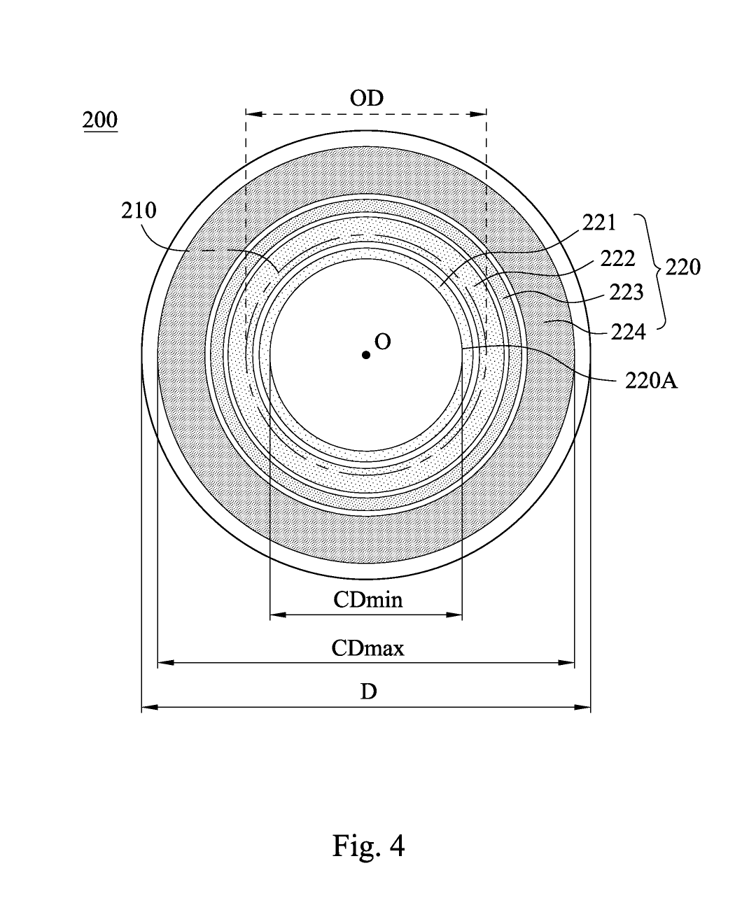

The light-blocking annular region 12 can include at least two is light-blocking parts (as shown in FIG. 4). The light-blocking parts can be adjacent but separately arranged to each other. Alternatively, the light-blocking parts can be adjacent and tightly connected to each other. Therefore, the amount of light passing through the contact lens 10 can be further reduced, and the effect for easing the photophobia can be enhanced.

The average transmittances of visible light of the light-blocking parts of the light-blocking annular region 12 can be gradually changed. That is, different light-blocking parts can have different color shades. The color shades of the light-blocking parts, from inside to outside, can be arranged from dark to light (Tavg1<Tavg2, wherein Tavg1 and Tavg2 are the average transmittances of visible light of the first light-blocking part and the second light-blocking part located from inside to outside, respectively). Alternatively, the color shades of the light-blocking parts, from inside to outside, can be arranged from light to dark (Tavg1>Tavg2). When there are more light-blocking parts, the color shades of the light-blocking parts, from inside to outside, can be arranged from dark to light to dark (Tavg1<Tavg2 and Tavg3<Tavg2, wherein Tavg1, Tavg2 and Tavg3 are the average transmittances of visible light of the first light-blocking part, the second light-blocking part and the third light-blocking part located from inside to outside, respectively). Alternatively, the color shades of the light-blocking parts, from inside to outside, can be arranged from light to dark to light (Tavg1>Tavg2 and Tavg3>Tavg2). Therefore, the effect of blocking visible light can be effectively controlled. The gradual change of the color shades can strengthen the blocking effect of certain regions on demands, meanwhile, the aesthetic requirement can be satisfied.

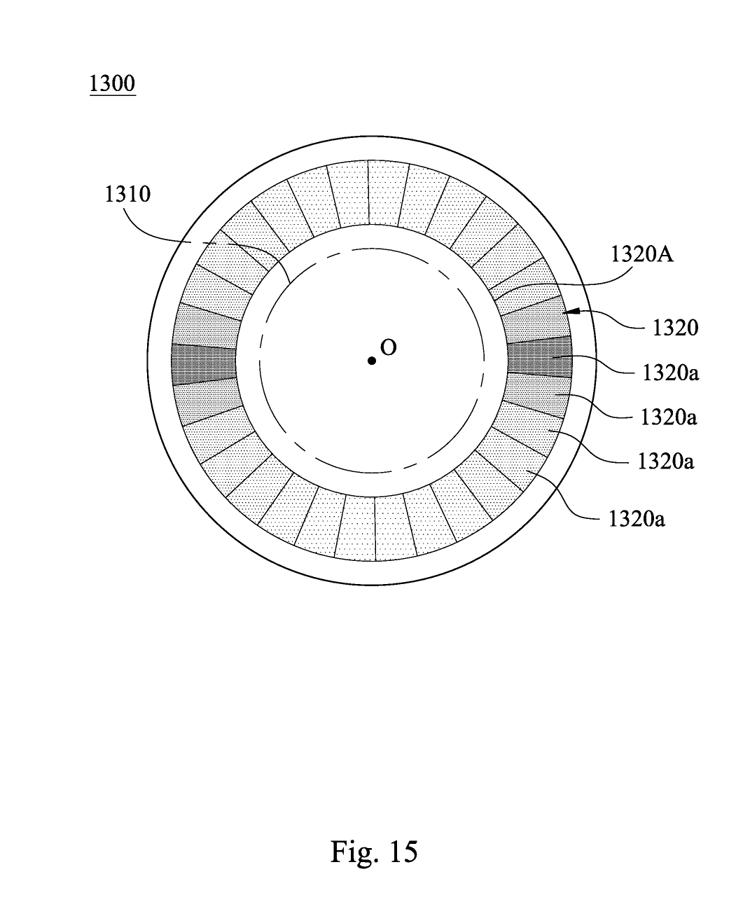

The light-blocking part of the light-blocking annular region 12 can include a plurality of sub light-blocking parts (as shown in FIG. 15). The sub light-blocking parts can be arranged in a radial pattern, and the average transmittances of visible light of the sub light-blocking parts can be gradually changed. That is, different sub light-blocking parts can have different color shades along the clockwise direction or along the counterclockwise direction. Therefore, the visual aesthetic effect can be enhanced, and the contact lens 10 can be featured with the recognition function of rotational positioning.

When a maximum diameter of the light-blocking annular region 12 is CDmax, and the minimum diameter of the light-blocking annular region 12 is CDmin, the following condition can be satisfied: 0.5 mm CDmax-CDmin≦13.0 mm. Therefore, the size of the light-blocking annular region 12 is proper, which can enhance the visual comfort and can satisfy the aesthetic requirement. Alternatively, the following condition can be satisfied: 1.0 mm CDmax-CDmin≦12.0 mm. Alternatively, the following condition can be satisfied: 1.5 mm CDmax-CDmin≦11.0 mm. Alternatively, the following condition can be satisfied: 2.0 mm≦CDmax-CDmin≦10.0 mm. Alternatively, the following condition can be satisfied: 3.0 mm CDmax-CDmin≦9.0 mm. Alternatively, the following condition can be satisfied: 3.0 mm≦CDmax-CDmin≦8.0 mm.

When the minimum diameter of the light-blocking annular region 12 is CDmin, and a maximum diameter of the optical region 11 is OD, the following condition can be satisfied: 0.05≦CDmin/OD≦2.0. Therefore, the amount of light passing through of the contact lens 10 is proper, which can ensure a proper brightness of the visual image. Alternatively, the following condition can be satisfied: 0.10≦CDmin/OD≦1.80. Alternatively, the following condition can be satisfied: 0.15≦CDmin/OD≦1.60. Alternatively, the following condition can be satisfied: 0.15≦CDmin/OD≦1.40. Alternatively, the following condition can be satisfied: 0.20≦CDmin/OD≦1.00. Alternatively, the following condition can be satisfied: 0.20≦CDmin/OD≦0.85.

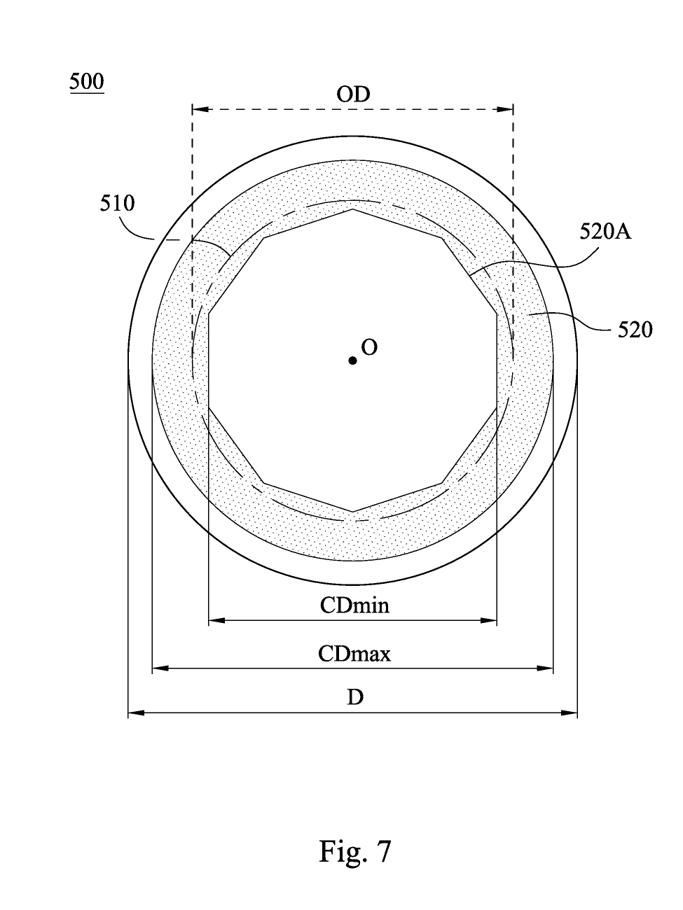

In the embodiment, an inner edge 12A of the light-blocking annular region 12 is formed in a circle. In other embodiment, the inner edge 12A of the light-blocking annular region 12 can be formed in a polygon with at least four sides (as shown in FIG. 7 to FIG. 10). Therefore, the inner edge 12A of the light-blocking annular region 12 can be designed according to the demands of the customers, which can prevent the patterns from monotone, and can provide the recognition function of rotational positioning for the contact lens for correcting the astigmatism or presbyopia or can provide the recognition function of rotational positioning for the multifocal contact lens.

The contact lens 10 can include a photochromic material. Therefore, it is favorable to reduce the irritation and injury caused by the strong light when in the outdoor environment. Moreover, the effect of blocking visible light of the light-blocking annular region 12 can be enhanced, and the applicability under environment with significant change of light intensity can be enhanced.

The photochromic material refers to a material (A) which can undergo a chemical reaction caused by the irradiation of light with a certain wavelength to range and is transformed into another material (B), and the material (B) can be reversibly transformed into the material (A) via light or heat. The material (A) and the material (B) have different absorption spectra, so that the colors of the material (A) and the material (B) are different. Specifically, the photochromic material can be selected, but is not limited to, from the group consisting of spiropyrans, spirooxazines, benzo and naphthopyrans, fulgides, diarylethenes and a combination thereof. The “combination thereof” refers that the photochromic material can include at least two of the spiropyrans, the spirooxazines, the benzo and naphthopyrans, the fulgides and the diarylethenes which are mixed in any ratio. For example, the spiropyrans can be, but are not limited to, 1′,3′-dihydro-1′,3′,3′-trimethyl-6-nitrospiro[2H-1-benzopyran-2,2′-(2H)-indole] (CAS No.: 1498-88-0) or 1′,3′-dihydro-8-methoxy-1′,3′,3′-trimethyl-6-nitrospiro[2H-1-benzopyran-2,2′-(2H)-indole] (CAS No.: 1498-89-1). The spirooxazines can be, but are not limited to, 1,3-dihydro-1,3,3-trimethylspiro[2H-indole-2,3′-[3H]haphth[2,1-b][1,4]oxazine] (CAS No.: 27333-47-7), 5-chloro-1,3-dihydro-1,3,3-trimethylspiro[2H-indole-2,3′-(3H)naphth[2,1-b](1,4)oxazine] (CAS No.: 27333-50-2) or 1,3-dihydro-1,3,3-trimethylspiro[2H-indole-2,3′-[3H]phenanthro[9,10-b](1,4)oxazine] (CAS No.: 119980-36-8). The benzo and naphthopyrans can be, but are not limited to, hydroxy-4-methyl-2H-naphtho(1,2-B)pyran-2-one (CAS No.: 21353-16-2). The fulgides can be, but are not limited to, (E)-benzylidene(diphenylmethylene)succinic anhydride (CAS No.: 37460-01-8) or to 3-[(E)-2-(2,5-dimethylfuran-3-yl)ethylidene]dihydro-4-(1-methylethylidene)-2,5-furandione (CAS No.: 77515-04-9). The diarylethenes can be, but are not limited to 9,10-dihydrophenanthrene (CAS No.: 776-35-2).

Specifically, at least one of the optical region 11 and the light-blocking annular region 12 includes the photochromic material, which means only the optical region 11 includes the photochromic material, or only the light-blocking annular region 12 includes the photochromic material, or both of the optical region 11 and the light-blocking annular region 12 include the photochromic material. When the optical region 11 includes the photochromic material, the contact lens can function as sunglasses in the outdoor environment with strong light, which can reduce the irritation and injury caused by the strong light. When the light-blocking annular region 12 includes the photochromic material, the effect of blocking visible light of the light-blocking annular region 12 can be enhanced, so that the contact lens 10 is suitable for both indoor environment and outdoor environment, and the applicability under environment with significant change of light intensity can be enhanced.

More specifically, the photochromic material can be added into the composition for manufacturing the contact lens 10, so that the entire contact lens 10 can provide the photochromic effect. Alternatively, the photochromic material can be only added into a portion of the contact lens 10. For example, the contact lens 10 can be a two-layer contact lens manufactured by a sandwich process. The two-layer contact lens includes two main structures, an object-side layer (not shown) and an image-side layer (not shown), and there is a color film (not shown) sandwiched between the object-side layer and the image-side layer. The color film can be formed on a surface of at least one of the object-side layer and the image-side layer via printing, vapor deposition or coating. Moreover, the color film can be formed on specific regions of the surface of at least one of the object-side layer and the image-side layer and can be formed in different patterns. In this case, the photochromic material can be added into the object-side layer and/or the image-side layer. For example, the photochromic material can be added into the composition for manufacturing the object-side layer and/or the image-side layer, so that the entire object-side layer and/or the entire image-side layer can provide the photochromic effect. Alternatively, the photochromic material can be (partially or completely) formed on the surface of the object-side layer and/or the image-side layer via printing, vapor deposition or coating. Alternatively, the photochromic material can be added into the color pigments of the color film, so that the photochromic material becomes a part of the color film and can be formed on specific regions of the surface of the object-side layer and/or the image-side layer so as to be formed in different patterns.

The contact lens 10 can be made of silicone hydrogel, and a composition for manufacturing the silicone hydrogel can include at least three kinds of monomers. The monomers can be selected from the group consisting of 2-hydroxyethyl methacrylate, methacrylic acid, glycerol monomethacrylate, N-vinyl-2-pyrrolidinone, N,N-dimethyl acrylamide, 3-methacryloyloxypropyltris(trimethylsilyloxy) silane, 3-(3-methacryloxy-2-hydroxypropoxy)propylbis(trimethylsiloxy)methylsilane, (3-acryloxy-2-hydroxypropoxypropyl) terminated polydimethylsiloxane and methyl methacrylate. In other words, at least three kinds of the to aforementioned monomers are selected as monomers of the composition for manufacturing the silicone hydrogel. Therefore, the oxygen permeability of the contact lens 10 can be enhanced, and the long wear comfort can be provided. The other ingredients of the composition for manufacturing the silicone hydrogel are conventional, and are not described herein.

The contact lens 10 can be made of hydrogel, and a composition for manufacturing the hydrogel can include at least two kinds of monomers. The monomers can be selected from the group consisting of 2-hydroxyethyl methacrylate, methacrylic acid, glycerol monomethacrylate, N-vinyl-2-pyrrolidinone, N,N-dimethyl acrylamide and methyl methacrylate. In other words, at least two kinds of the aforementioned monomers are selected as monomers of the composition for manufacturing the hydrogel. Therefore, the water content of the contact lens 10 and the comfort provided thereby can be enhanced. Accordingly, the moisture, the smoothness and the softness of the contact lens 10 can be maintained, and the foreign body sensation can be avoided. The other ingredients of the composition for manufacturing the hydrogel are conventional, and are not described herein.

The contact lens 10 can be a multifocal contact lens. Therefore, it is favorable for correcting astigmatism, presbyopia, myopia and hyperopia. When the multifocal contact lens is applied to correct myopia, a vision control effect for slowing down the development of myopia can be provided.

When the maximum diameter of the light-blocking annular region 12 is CDmax, and the maximum diameter of the contact lens is D, the following condition can be satisfied: 0.5≦CDmax/D≦1.0. Therefore, an enlarged effect to of the pupil area can be provided in the outdoor environment, and can reduce the injury caused by the strong light in the outdoor environment. Alternatively, the following condition can be satisfied: 0.55≦CDmax/D≦1.00. Alternatively, the following condition can be satisfied: 0.65≦CDmax/D≦1.00. Alternatively, the following condition can be satisfied: 0.75≦CDmax/D≦1.00. Alternatively, is the following condition can be satisfied: 0.80 CDmax/D≦0.95.

FIG. 1B is a side view of the contact lens 10 in FIG. 1A. As shown in FIG. 1B, when a base curve of the contact lens 10 is BC, the following condition can be satisfied: 9.00 mm≦BC. Therefore, the base curve with a larger curvature radius can be provided, which is favorable for fitting a relatively flat cornea, so that the wear comfort and fitting degree for the relatively flat cornea can be enhanced. For example, a person undergone a laser corneal surgery has a relatively flat cornea due to some of the cornea has been removed. The base curve of conventional contact lens cannot fit well with the relatively flat cornea and thus fails to provide the desired wear comfort and fitness. Therefore, the contact lens 10 according to the present disclosure is suitable for people who have undergone the laser corneal surgery. Alternatively, the following condition can be satisfied: 9.25 mm≦BC. Alternatively, the following condition can be satisfied: 9.50 mm≦BC. Alternatively, the following condition can be satisfied: 9.75 mm≦BC. Alternatively, the following condition can be satisfied: 10.00 mm≦BC≦100.00 mm.

Specifically, the base curve refers to a curve of a back surface (its reference numeral is omitted) of the contact lens 10 towards the cornea. When the base curve is larger, the back surface of the contact lens 10 is more flat. When the base curve of the contact lens 10 is larger than a base curve of to an eyeball, the contact lens 10 cannot fit well with the surface of the eyeball and tends to shift on the surface of the eyeball. When the base curve of the contact lens 10 is smaller than the base curve of the eyeball, the stress and discomfort may occur. Therefore, a significant difference between the base curve of the contact lens 10 and the base curve of the eyeball tends to cause is the vision fluctuation or the falling of the contact lens 10, and even worse, the corneal infection may occur. In general, the base curve of the contact lens 10 is designed larger than the base curve of the eyeball about 0.4 mm. The base curves of the eyeballs of Chinese people are about 8.4 mm to 8.6 mm, so that the base curves of the commercial available contact lenses are normally less than 9 mm, which are not suitable for the relative flat corneas of the people who have undergone the laser corneal surgery.

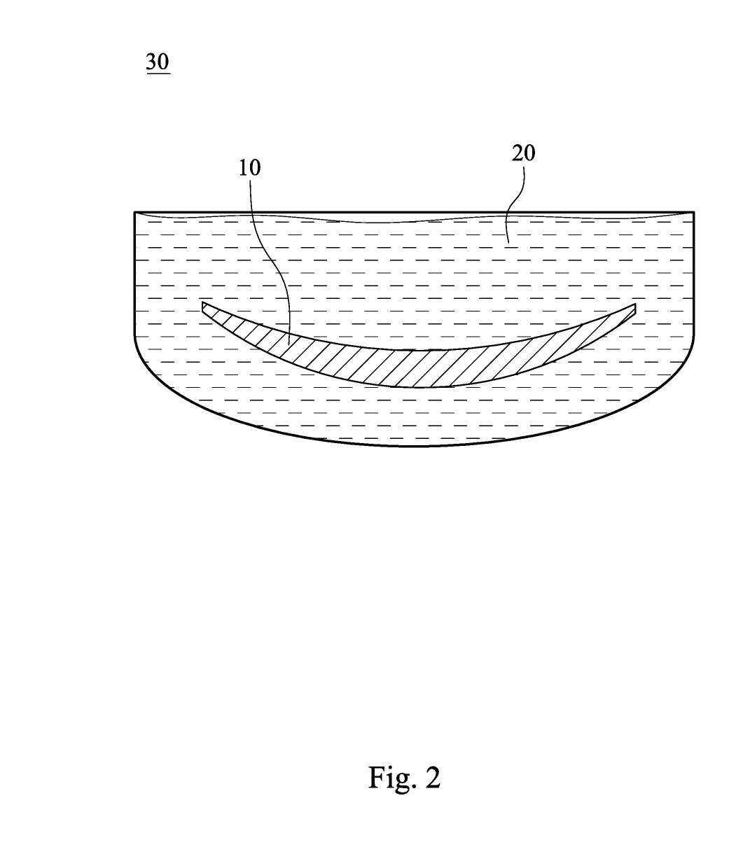

FIG. 2 is a schematic view of a contact lens product 30 according to another embodiment of the present disclosure. In FIG. 2, the contact lens product 30 includes the contact lens 10 and a soaking solution 20, wherein the contact lens 10 is immersed in the soaking solution 20. The details of the contact lens 10 have been mentioned above, and will not be repeated herein. The soaking solution 20 can be a commercially available solution for immersing and preserving contact lenses.

At least one of the contact lens 10 and the soaking solution 20 can include a cycloplegic agent, which means only the contact lens 10 includes the cycloplegic agent, or only the soaking solution 20 includes the cycloplegic agent, or both of the contact lens 10 and the soaking solution 20 include cycloplegic agent. Therefore, the peripheral aberrations and image blur due to the enlarged pupil caused by the cycloplegic agent can be improved by the contact to lens 10 according to the present disclosure. In general, the pupil is enlarged 2 times to 4 times by the cycloplegic agent. When the pupil is enlarged, an excessive amount light is allowed to enter into the eyes, which interferes the imaging and results in the peripheral aberrations. However, according to the contact lens 10 of the present disclosure, the excessive amount of light passing is through the contact lens 10 can be prevented meanwhile the amount of light is sufficient, which can avoid the photophobia and eliminate the peripheral aberrations. Accordingly, the clearness and the sharpness of the image of the visual field can be enhanced.

Specifically, the cycloplegic agent can be added into the composition for manufacturing the contact lens 10, so that the cycloplegic agent is contained inside the contact lens 10. When wearing the contact lens 10, the cycloplegic agent is slowly released, which can slow down the development of myopia. The patient can control the vision via simply wearing the contact lens 10, which can simplify the correction of the myopia. Alternatively, the cycloplegic agent can be added into to a commercially available solution for immersing and preserving contact lenses so as to obtain the soaking solution 20 including the cycloplegic agent. Therefore, the cycloplegic agent can function immediately when wearing the contact lens 10 taken from the soaking solution 20. The effect is immediate and the correction of the myopia can also be simplified. When a weight percentage concentration of the cycloplegic agent in the contact lens 10 or in the soaking solution 20 is ConA, the following condition can be satisfied: 0%<ConA≦1%. Alternatively, the following condition can be satisfied: 0%<ConA≦0.5%. Alternatively, the following condition can be satisfied: 0%<ConA≦0.25%. Alternatively, the following condition can be to satisfied: 0%<ConA≦0.1%. Alternatively, the following condition can be satisfied: 0%<ConA≦0.05%. Alternatively, the following condition can be satisfied: 0%<ConA≦0.01%.

According to the above description of the present disclosure, the following 1st-13th specific examples are provided for further explanation.

### 1st Example

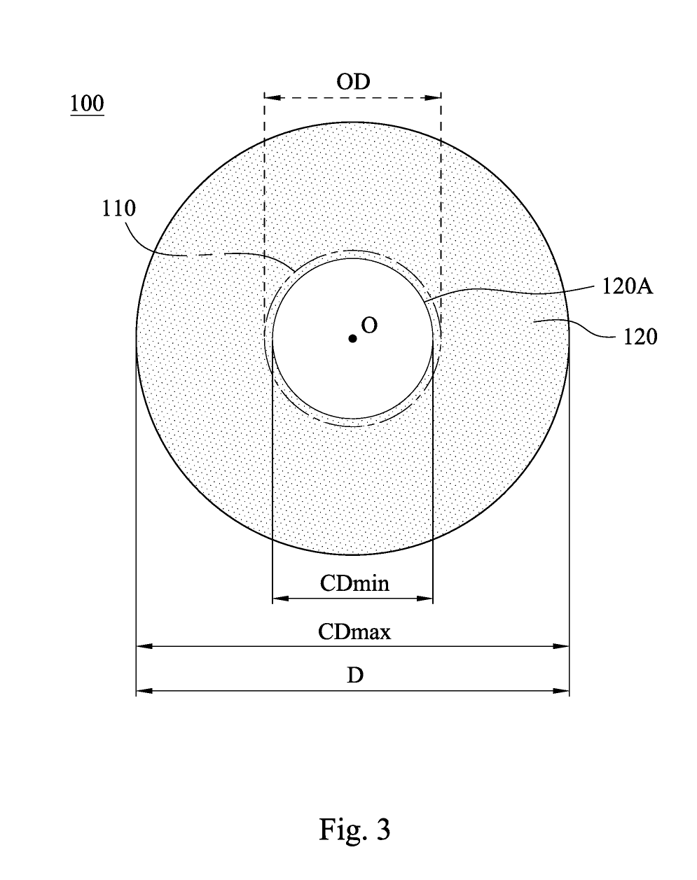

FIG. 3 is a schematic view of a contact lens 100 according to the 1st example of the present disclosure. In FIG. 3, the contact lens 100 includes an optical region 110 and a light-blocking annular region 120. The light-blocking annular region 120 surrounds a central point O of the contact lens 100, and an inner edge 120A of the light-blocking annular region 120 is formed in a circle. In the 1st example, the optical region 110 is the region circled by the dashed line, and the optical region 110 and the light-blocking annular region 120 are partially overlapped.

In the 1st example, a minimum diameter of the light-blocking annular region 120 is CDmin, a maximum diameter of the light-blocking annular region 120 is CDmax, a maximum diameter of the optical region 110 is OD, a maximum diameter of the contact lens 100 is D, a base curve of the contact lens 100 is BC (which is illustrated in FIG. 1A). The values of CDmin, CDmax, OD, D, CDmin/D, CDmax/D, CDmax-CDmin, CDmin/OD and BC of the 1st example are listed in Table 1.

TABLE 1

| | | | |
|---|---|---|---|
| CDmin (mm) | 5.00 | CDmax/D | 1.00 |
| CDmax (mm) | 13.50 | CDmax − CDmin | 8.50 |
| OD (mm) | 5.50 | CDmin/OD | 0.91 |
| D (mm) | 13.50 | BC (mm) | 8.00 |
| CDmin/D | 0.37 | | |

The contact lens 100 is made of hydrogel, and a composition for to manufacturing the hydrogel includes the following monomers: 2-hydroxyethyl methacrylate, methacrylic acid, glycerol monomethacrylate and N-vinyl-2-pyrrolidinone. The contact lens 100 is a monofocal contact lens.

An average transmittance of visible light of the light-blocking annular region 120 is Tavg. The light-blocking annular region 120 is irradiated with a is light in a wavelength range of 200 nm to 400 nm for 10 seconds. The values of Tavg before and after irradiating with the light in the wavelength range of 200 nm to 400 nm are listed in Table 2.

TABLE 1

| | |
|---|---|
| (before) Tavg (%) | 8 |
| (after) Tavg (%) | 8 |

### 2nd Example

FIG. 4 is a schematic view of a contact lens 200 according to the 2nd example of the present disclosure. In FIG. 4, the contact lens 200 includes an optical region 210 and a light-blocking annular region 220. The light-blocking annular region 220 surrounds a central point O of the contact lens 200, and an inner edge 220A of the light-blocking annular region 220 is formed in a circle. Specifically, the light-blocking annular region 220 includes four disconnected light-blocking parts which are the first light-blocking part 221, the second light-blocking part 222, the third light-blocking part 223 and the fourth light-blocking part 224. In the 2nd example, the optical region 210 is the region circled by the dashed line, and the optical region 210 and the light-blocking annular region 220 are partially overlapped.

The values of CDmin, CDmax, OD, D, CDmin/D, CDmax/D, CDmax-CDmin, CDmin/OD and BC of the 2nd example are listed in Table 3. The definitions of these parameters shown in Table 3 are the same as those is stated in the 1st example, so an explanation in this regard will not be provided again.

TABLE 3

| | | | |
|---|---|---|---|
| CDmin (mm) | 6.00 | CDmax/D | 0.93 |
| CDmax (mm) | 13.00 | CDmax − CDmin | 7.00 |
| OD (mm) | 7.50 | CDmin/OD | 0.80 |
| D (mm) | 14.00 | BC (mm) | 8.75 |
| CDmin/D | 0.43 | | |

The contact lens 200 is made of hydrogel, and a composition for manufacturing the hydrogel includes the following monomers: 2-hydroxyethyl methacrylate, methacrylic acid and glycerol monomethacrylate. The contact lens 200 is a monofocal contact lens.

An average transmittance of visible light of the light-blocking annular region 220 is Tavg. The light-blocking annular region 220 is irradiated with a light in a wavelength range of 200 nm to 400 nm for 10 seconds. The values of Tavg before and after irradiating with the light in the wavelength range of 200 nm to 400 nm are listed in Table 4.

TABLE 4

| | |
|---|---|
| (before) Tavg (%) | 27 |
| (after) Tavg (%) | 27 |

Moreover, the light-blocking annular region 220 includes, from inside to outside, the first light-blocking part 221, the second light-blocking part 222, the third light-blocking part 223 and the fourth light-blocking part 224, which can have different average transmittances of visible light under the same condition. In the 2nd example, when an average transmittance of visible light of the first light-blocking part 221 is Tavg1, an average transmittance of visible light of the second light-blocking part 222 is Tavg2, an average transmittance of visible light of the third light-blocking part 223 is Tavg3, and an average transmittance of visible light of the fourth light-blocking part 224 is Tavg4, the following condition is satisfied: Tavg1>Tavg2>Tavg3>Tavg4 (under the same condition). Table 4 shows the data of the light-blocking part (at least one of the light-blocking parts 221-224) having the minimal average transmittance of visible light, which can provide the best effect for blocking visible light. In the 2nd example, the following condition is satisfied: Tavg=Tavg4.

### 3rd Example

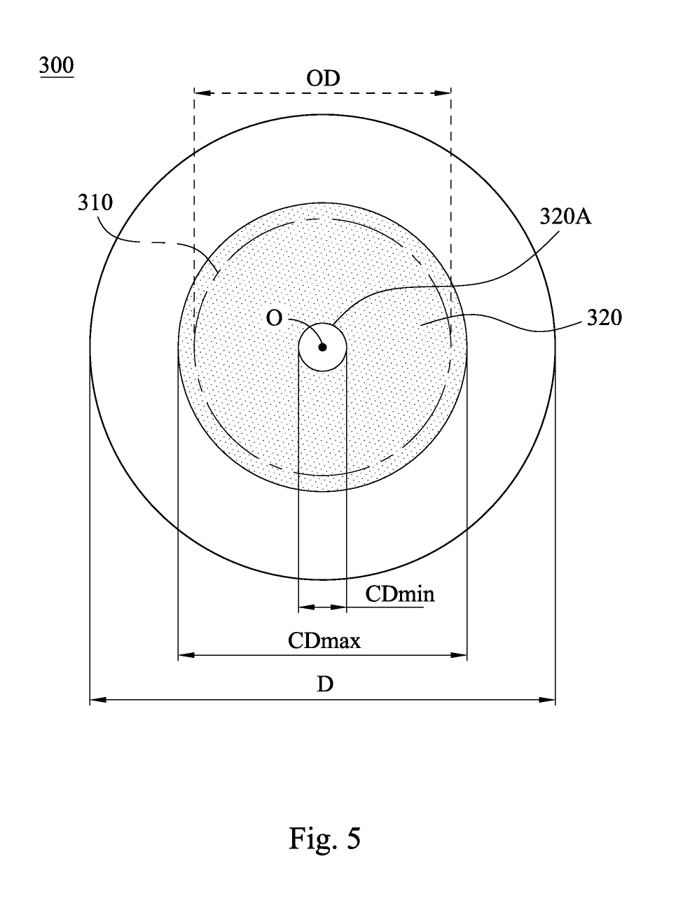

FIG. 5 is a schematic view of a contact lens 300 according to the 3rd example of the present disclosure. In FIG. 5, the contact lens 300 includes an optical region 310 and a light-blocking annular region 320. The light-blocking annular region 320 surrounds a central point O of the contact lens 300, and an inner edge 320A of the light-blocking annular region 320 is formed in a circle. In the 3rd example, the optical region 310 is the region circled by the dashed line, and the optical region 310 and the light-blocking annular region 320 are partially overlapped.

The values of CDmin, CDmax, OD, D, CDmin/D, CDmax/D, to CDmax-CDmin, CDmin/OD and BC of the 3rd example are listed in Table 5. The definitions of these parameters shown in Table 5 are the same as those stated in the 1st example, so an explanation in this regard will not be provided again.

TABLE 5

| | | | |
|---|---|---|---|
| CDmin (mm) | 1.50 | CDmax/D | 0.62 |
| CDmax (mm) | 9.00 | CDmax − CDmin | 7.50 |
| OD (mm) | 8.00 | CDmin/OD | 0.19 |
| D (mm) | 14.50 | BC (mm) | 8.50 |
| CDmin/D | 0.10 | | |

The contact lens 300 is made of hydrogel, and a composition for manufacturing the hydrogel includes the following monomers: 2-hydroxyethyl methacrylate and methacrylic acid. The contact lens 300 is a multifocal contact lens.

An average transmittance of visible light of the light-blocking annular region 320 is Tavg. The light-blocking annular region 320 is irradiated with a light in a wavelength range of 200 nm to 400 nm for 10 seconds. The values of Tavg before and after irradiating with the light in the wavelength range of 200 nm to 400 nm are listed in Table 6.

TABLE 6

| | |
|---|---|
| (before) Tavg (%) | 55 |
| (after) Tavg (%) | 55 |

### 4th Example

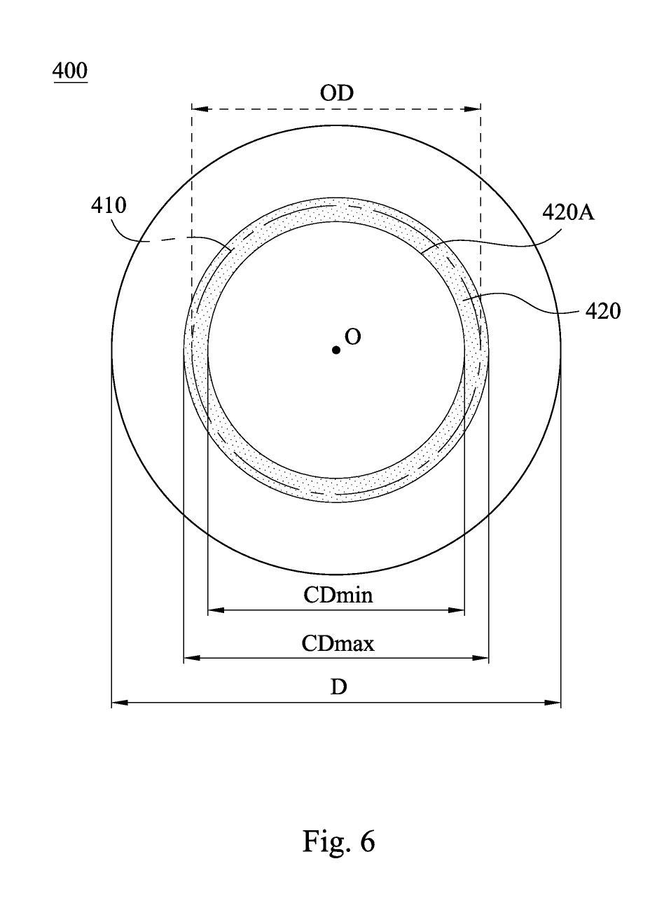

FIG. 6 is a schematic view of a contact lens 400 according to the 4th example of the present disclosure. In FIG. 6, the contact lens 400 includes an optical region 410 and a light-blocking annular region 420. The light-blocking annular region 420 surrounds a central point O of the contact lens 400, and an inner edge 420A of the light-blocking annular region 420 is formed in a circle. In the 4th example, the optical region 410 is the region circled by the dashed line, and the optical region 410 and the light-blocking annular region 420 are partially overlapped.

The values of CDmin, CDmax, OD, D, CDmin/D, CDmax/D, CDmax-CDmin, CDmin/OD and BC of the 4th example are listed in Table 7. The definitions of these parameters shown in Table 7 are the same as those stated in the 1st example, so an explanation in this regard will not be provided again.

TABLE 7

| | | | |
|---|---|---|---|
| CDmin (mm) | 8.00 | CDmax/D | 0.68 |
| CDmax (mm) | 9.50 | CDmax − CDmin | 1.50 |
| OD (mm) | 9.00 | CDmin/OD | 0.89 |
| D (mm) | 14.00 | BC (mm) | 9.00 |
| CDmin/D | 0.57 | | |

The contact lens 400 is made of hydrogel, and a composition for manufacturing the hydrogel includes the following monomers: 2-hydroxyethyl methacrylate and glycerol monomethacrylate. The contact lens 400 is a multifocal contact lens, and the contact lens 400 includes a cycloplegic agent.

An average transmittance of visible light of the light-blocking annular region 420 is Tavg. The light-blocking annular region 420 is irradiated with a light in a wavelength range of 200 nm to 400 nm for 10 seconds. The values of Tavg before and after irradiating with the light in the wavelength range of 200 nm to 400 nm are listed in Table 8.

TABLE 8

| | |
|---|---|
| (before) Tavg (%) | 35 |
| (after) Tavg (%) | 35 |

### 5th Example

FIG. 7 is a schematic view of a contact lens 500 according to the 5th example of the present disclosure. In FIG. 7, the contact lens 500 includes an optical region 510 and a light-blocking annular region 520. The light-blocking annular region 520 surrounds a central point O of the contact lens 500, and an inner edge 520A of the light-blocking annular region 520 is formed in a decagon. In the 5th example, the optical region 510 is the region circled by the dashed line, and the optical region 510 and the light-blocking annular region 520 are partially overlapped.

The values of CDmin, CDmax, OD, D, CDmin/D, CDmax/D, CDmax-CDmin, CDmin/OD and BC of the 5th example are listed in Table 9. The definitions of these parameters shown in Table 9 are the same as those stated in the 1st Example, so an explanation in this regard will not be provided again.

TABLE 9

| | | | |
|---|---|---|---|
| CDmin (mm) | 9.00 | CDmax/D | 0.89 |
| CDmax (mm) | 12.50 | CDmax − CDmin | 3.50 |
| OD (mm) | 10.00 | CDmin/OD | 0.90 |
| D (mm) | 14.00 | BC (mm) | 9.25 |
| CDmin/D | 0.64 | | |

The contact lens 500 is made of hydrogel, and a composition for manufacturing the hydrogel includes the following monomers: 2-hydroxyethyl methacrylate and N-vinyl-2-pyrrolidinone. The contact lens 500 is a monofocal contact lens.

An average transmittance of visible light of the light-blocking annular region 520 is Tavg. The light-blocking annular region 520 is irradiated with a light in a wavelength range of 200 nm to 400 nm for 10 seconds. The values of Tavg before and after irradiating with the light in the wavelength range of 200 nm to 400 nm are listed in Table 10.

TABLE 10

| | |
|---|---|
| (before) Tavg (%) | 15 |
| (after) Tavg (%) | 15 |

### 6th Example

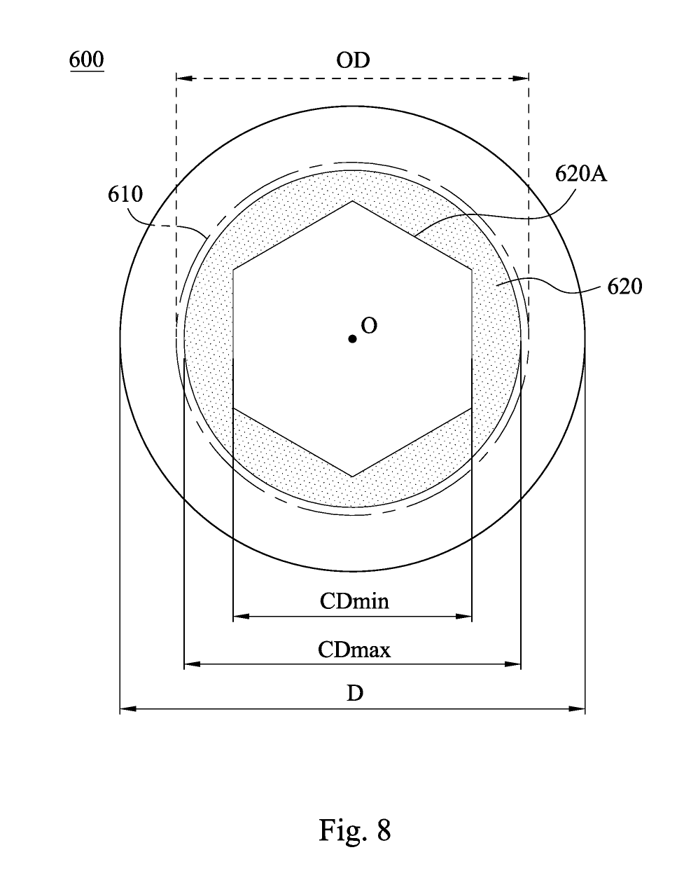

FIG. 8 is a schematic view of a contact lens 600 according to the 6th example of the present disclosure. In FIG. 8, the contact lens 600 includes an is optical region 610 and a light-blocking annular region 620. The light-blocking annular region 620 surrounds a central point O of the contact lens 600, and an inner edge 620A of the light-blocking annular region 620 is formed in a hexagon. In the 6th example, the optical region 610 is the region circled by the dashed line, and the optical region 610 and the light-blocking annular region 620 are partially overlapped.

The values of CDmin, CDmax, OD, D, CDmin/D, CDmax/D, CDmax-CDmin, CDmin/OD and BC of the 6th example are listed in Table 11. The definitions of these parameters shown in Table 11 are the same as those stated in the 1st Example, so an explanation in this regard will not be provided again.

TABLE 11

| | | | |
|---|---|---|---|
| CDmin (mm) | 7.50 | CDmax/D | 0.72 |
| CDmax (mm) | 10.50 | CDmax-CDmin | 3.00 |
| OD (mm) | 11.00 | CDmin/OD | 0.68 |
| D (mm) | 14.50 | BC (mm) | 9.50 |
| CDmin/D | 0.52 | | |

The contact lens 600 is made of hydrogel, and a composition for manufacturing the hydrogel includes the following monomers: 2-hydroxyethyl methacrylate, glycerol monomethacrylate and N-vinyl-2-pyrrolidinone. The to contact lens 600 is a monofocal contact lens, and the contact lens 600 includes a cycloplegic agent.

The light-blocking annular region 620 includes a photochromic material. In the 6th example, the photochromic material includes 1′,3′-dihydro-1′,3′,3′-trimethyl-6-nitrospiro[2H-1-benzopyran-2,2′-(2H)-indole]. An average transmittance of visible light of the light-blocking annular region 620 is Tavg. The light-blocking annular region 620 is irradiated with a light in a wavelength range of 200 nm to 400 nm for 10 seconds. The values of Tavg before and after irradiating with the light in the wavelength range of 200 nm to 400 nm are listed in Table 12.

TABLE 12

| | |
|---|---|
| (before) Tavg (%) | 40 |
| (after) Tavg (%) | 10 |

### 7th Example

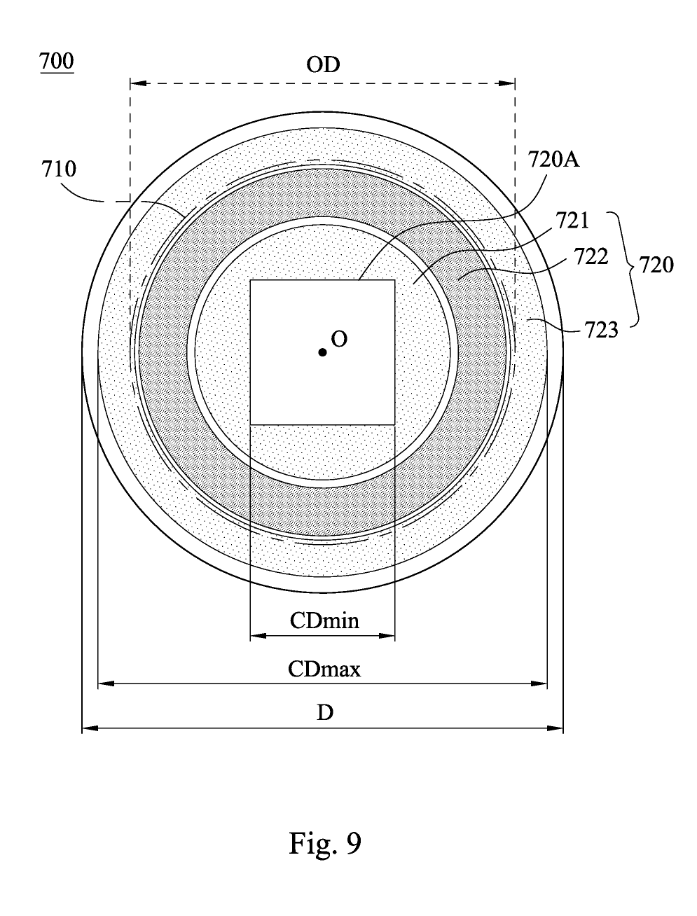

FIG. 9 is a schematic view of a contact lens 700 according to the 7th example of the present disclosure. In FIG. 9, the contact lens 700 includes an optical region 710 and a light-blocking annular region 720. The light-blocking annular region 720 surrounds a central point O of the contact lens 700, and an inner edge 720A of the light-blocking annular region 720 is formed in a quadrilateral. Specifically, the light-blocking annular region 720 includes three disconnected light-blocking parts which are the first light-blocking part 721, the second light-blocking part 722 and the third light-blocking part 723. In the 7th example, the optical region 710 is the region circled by the dashed line, and the optical region 710 and the light-blocking annular region 720 are partially overlapped.

The values of CDmin, CDmax, OD, D, CDmin/D, CDmax/D, CDmax-CDmin, CDmin/OD and BC of the 7th example are listed in Table 13. The definitions of these parameters shown in Table 13 are the same as those stated in the 1st Example, so an explanation in this regard will not be provided again.

TABLE 13

| | | | |
|---|---|---|---|
| CDmin (mm) | 4.50 | CDmax/D | 0.93 |
| CDmax (mm) | 14.00 | CDmax-CDmin | 9.50 |
| OD (mm) | 12.00 | CDmin/OD | 0.38 |
| D (mm) | 15.00 | BC (mm) | 10.00 |
| CDmin/D | 0.30 | | |

The contact lens 700 is made of hydrogel, and a composition for manufacturing the hydrogel includes the following monomers: 2-hydroxyethyl methacrylate, methacrylic acid and N-vinyl-2-pyrrolidinone. The contact lens 700 is a multifocal contact lens.

The light-blocking annular region 720 includes a photochromic material. In the 7th example, the photochromic material includes 1,3-dihydro-1,3,3-trimethylspiro[2H-indole-2,3′-[3H]naphth[2,1-b][1,4]oxazine]. An average transmittance of visible light of the light-blocking annular region 720 is Tavg. The light-blocking annular region 720 is irradiated with a light in a wavelength range of 200 nm to 400 nm for 10 seconds. The values of Tavg before and after irradiating with the light in the wavelength range of 200 nm to 400 nm are listed in Table 14.

TABLE 14

| | |
|---|---|
| (before) Tavg (%) | 55 |
| (after) Tavg (%) | 8 |

Moreover, the light-blocking annular region 720 includes, from inside to outside, the first light-blocking part 721, the second light-blocking part 722 and the third light-blocking part 723, which can have different average transmittances of visible light under the same condition. In the 7th example, when an average transmittance of visible light of the first light-blocking part 721 is Tavg1, an average transmittance of visible light of the second light-blocking part 722 is Tavg2, and an average transmittance of visible light of the third light-blocking part 723 is Tavg3, the following conditions are satisfied: Tavg1>Tavg2; and Tavg3>Tavg2 (under the same condition). Tavg1 and Tavg3 can be identical or different. Table 14 shows the data of the light-blocking part (at least one of the light-blocking parts 721-723) having the minimal average transmittance of visible light, which can provide the best effect for blocking visible light. In the 7th example, the following condition is satisfied: Tavg=Tavg2.

### 8th Example

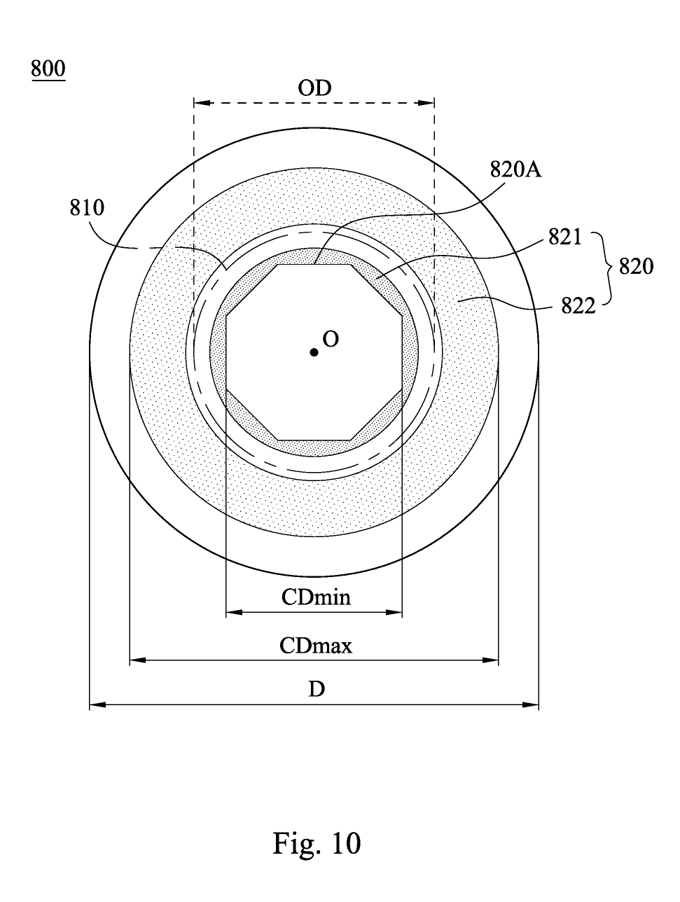

FIG. 10 is a schematic view of a contact lens 800 according to the 8th example of the present disclosure. In FIG. 10, the contact lens 800 includes an optical region 810 and a light-blocking annular region 820. The light-blocking annular region 820 surrounds a central point O of the contact lens 800, and an inner edge 820A of the light-blocking annular region 820 is formed in an octagon. Specifically, the light-blocking annular region 820 includes two disconnected light-blocking parts which are the first light-blocking part 821 and the second light-blocking part 822. In the 8th example, the optical region 810 is the region circled by the dashed line, and the optical region 810 and the light-blocking annular region 820 are partially overlapped.

The values of CDmin, CDmax, OD, D, CDmin/D, CDmax/D, CDmax-CDmin, CDmin/OD and BC of the 8th example are listed in Table 15. The definitions of these parameters shown in Table 15 are the same as those stated in the 1st Example, so an explanation in this regard will not be provided again.

TABLE 15

| | | | |
|---|---|---|---|
| CDmin (mm) | 5.50 | CDmax/D | 0.82 |
| CDmax (mm) | 11.50 | CDmax-CDmin | 6.00 |
| OD (mm) | 7.50 | CDmin/OD | 0.73 |
| D (mm) | 14.00 | BC (mm) | 10.50 |
| CDmin/D | 0.39 | | |

The contact lens 800 is made of silicone hydrogel, and a composition for manufacturing the silicone hydrogel includes the following monomers: 2-hydroxyethyl methacrylate, N-vinyl-2-pyrrolidinone, N,N-dimethyl acrylamide, (3-acryloxy-2-hydroxypropoxypropyl) terminated polydimethylsiloxane and methyl methacrylate. The contact lens 800 is a multifocal contact lens.

The light-blocking annular region 820 includes a photochromic material. In the 8th example, the photochromic material includes hydroxy-4-methyl-2H-naphtho(1,2-B)pyran-2-one. An average transmittance of visible light of the light-blocking annular region 820 is Tavg. The light-blocking annular region 820 is irradiated with a light in a wavelength range of 200 nm to 400 nm for 10 seconds. The values of Tavg before and after irradiating with the light in the wavelength range of 200 nm to 400 nm are listed in Table 16.

TABLE 16

| | |
|---|---|
| (before) Tavg (%) | 25 |
| (after) Tavg (%) | 11 |

Moreover, the light-blocking annular region 820 includes, from inside to outside, the first light-blocking part 821 and the second light-blocking part 822, which can have different average transmittances of visible light under the same condition. In the 8th example, when an average transmittance of visible light of the first light-blocking part 821 is Tavg1, and an average transmittance of visible light of the second light-blocking part 822 is Tavg2, the following condition is satisfied: Tavg1<Tavg2 (under the same condition). Table 16 shows the data of the light-blocking part (at least one of the light-blocking parts 821-822) having the minimal average transmittance of visible light, which can provide the best effect for blocking visible light. In the 8th example, the following condition is satisfied: Tavg=Tavg1.

### 9th Example

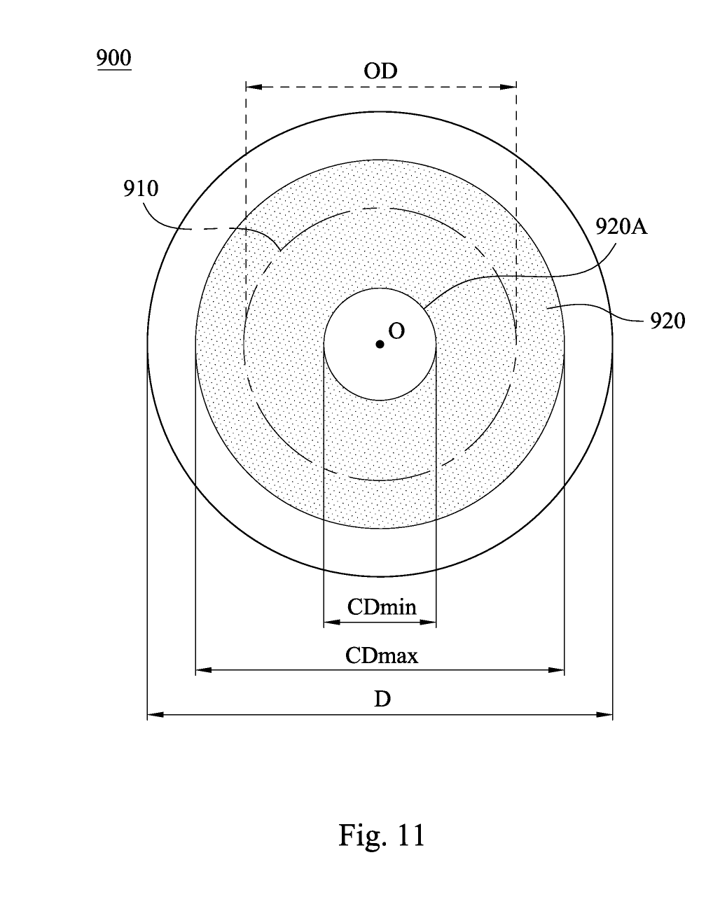

FIG. 11 is a schematic view of a contact lens 900 according to the 9th example of the present disclosure. In FIG. 11, the contact lens 900 includes an optical region 910 and a light-blocking annular region 920. The light-blocking annular region 920 surrounds a central point O of the contact lens 900, and an inner edge 920A of the light-blocking annular region 920 is formed in a circle. In the 9th example, the optical region 910 is the region circled by the dashed line, and the optical region 910 and the light-blocking annular region 920 are partially overlapped.

The values of CDmin, CDmax, OD, D, CDmin/D, CDmax/D, CDmax-CDmin, CDmin/OD and BC of the 9th example are listed in Table 17. The definitions of these parameters shown in Table 17 are the same as those stated in the 1st Example, so an explanation in this regard will not be provided again.

TABLE 17

| | | | |
|---|---|---|---|
| CDmin (mm) | 3.50 | CDmax/D | 0.79 |
| CDmax (mm) | 11.50 | CDmax-CDmin | 8.00 |
| OD (mm) | 8.50 | CDmin/OD | 0.41 |
| D (mm) | 14.50 | BC (mm) | 9.75 |
| CDmin/D | 0.24 | | |

The contact lens 900 is made of silicone hydrogel, and a composition for manufacturing the silicone hydrogel includes the following monomers: 2-hydroxyethyl methacrylate, 3-methacryloyloxypropyltris(trimethylsilyloxy) silane, 3-(3-methacryloxy-2-hydroxypropoxy)propylbis(trimethylsiloxy)methylsilane and methyl methacrylate. The contact lens 900 is a monofocal contact lens.

The light-blocking annular region 920 includes a photochromic material. In the 9th example, the photochromic material includes (E)-benzylidene(diphenylmethylene)succinic anhydride. An average transmittance of visible light of the light-blocking annular region 920 is Tavg. The light-blocking annular region 920 is irradiated with a light in a wavelength range of 200 nm to 400 nm for 10 seconds. The values of Tavg before and after irradiating with the light in the wavelength range of 200 nm to 400 nm are listed in Table 18.

TABLE 18

| | |
|---|---|
| (before) Tavg (%) | 20 |
| (after) Tavg (%) | 15 |

### 10th Example

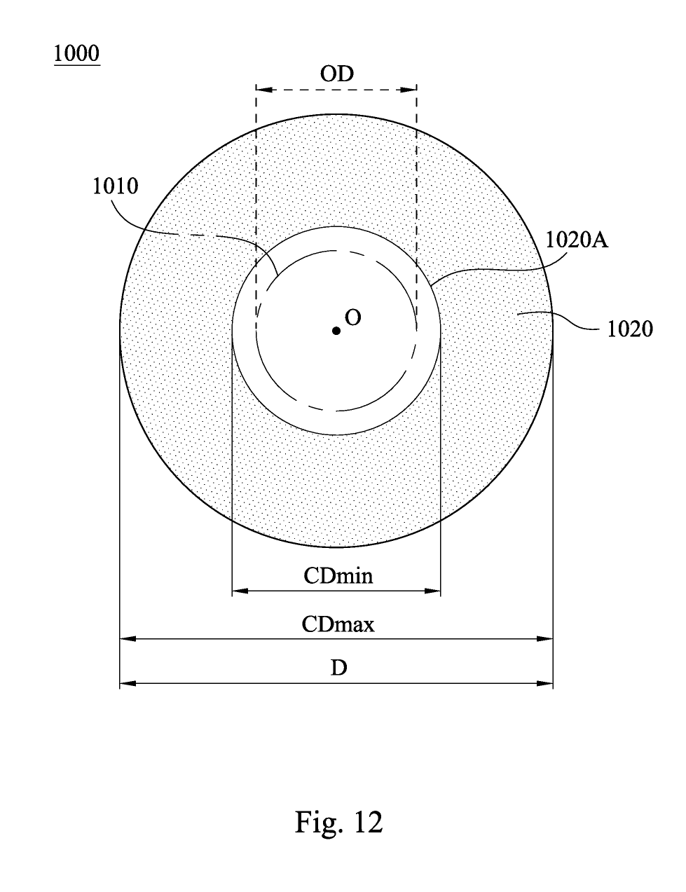

FIG. 12 is a schematic view of a contact lens 1000 according to the 10th example of the present disclosure. In FIG. 12, the contact lens 1000 includes an optical region 1010 and a light-blocking annular region 1020. The light-blocking annular region 1020 surrounds a central point O of the contact is lens 1000, and an inner edge 1020A of the light-blocking annular region 1020 is formed in a circle. In the 10th example, the optical region 1010 is the region circled by the dashed line, and the optical region 1010 and the light-blocking annular region 1020 are staggered.

The values of CDmin, CDmax, OD, D, CDmin/D, CDmax/D, CDmax-CDmin, CDmin/OD and BC of the 10th example are listed in Table 19. The definitions of these parameters shown in Table 19 are the same as those stated in the 1st Example, so an explanation in this regard will not be provided again.

TABLE 19

| | | | |
|---|---|---|---|
| CDmin (mm) | 6.50 | CDmax/D | 1.00 |
| CDmax (mm) | 13.50 | CDmax-CDmin | 7.00 |
| OD (mm) | 5.00 | CDmin/OD | 1.30 |
| D (mm) | 13.50 | BC (mm) | 12.00 |
| CDmin/D | 0.48 | | |

The contact lens 1000 is made of silicone hydrogel, and a composition for manufacturing the silicone hydrogel includes the following monomers: 2-hydroxyethyl methacrylate, N-vinyl-2-pyrrolidinone, N,N-dimethyl acrylamide, 3-methacryloyloxypropyltris(trimethylsilyloxy) silane, (3-acryloxy-2-hydroxypropoxypropyl) terminated polydimethylsiloxane and methyl methacrylate. The contact lens 1000 is a monofocal contact lens, and the contact lens 1000 includes a cycloplegic agent.

The light-blocking annular region 1020 includes a photochromic material. In the 10th example, the photochromic material includes 9,10-dihydrophenanthrene. An average transmittance of visible light of the light-blocking annular region 1120 is Tavg. The light-blocking annular region 1020 is irradiated with a light in a wavelength range of 200 nm to 400 nm for 10 seconds. The values of Tavg before and after irradiating with the light in the is wavelength range of 200 nm to 400 nm are listed in Table 20.

TABLE 20

| | |
|---|---|
| (before) Tavg (%) | 30 |
| (after) Tavg (%) | 13 |

### 11th Example

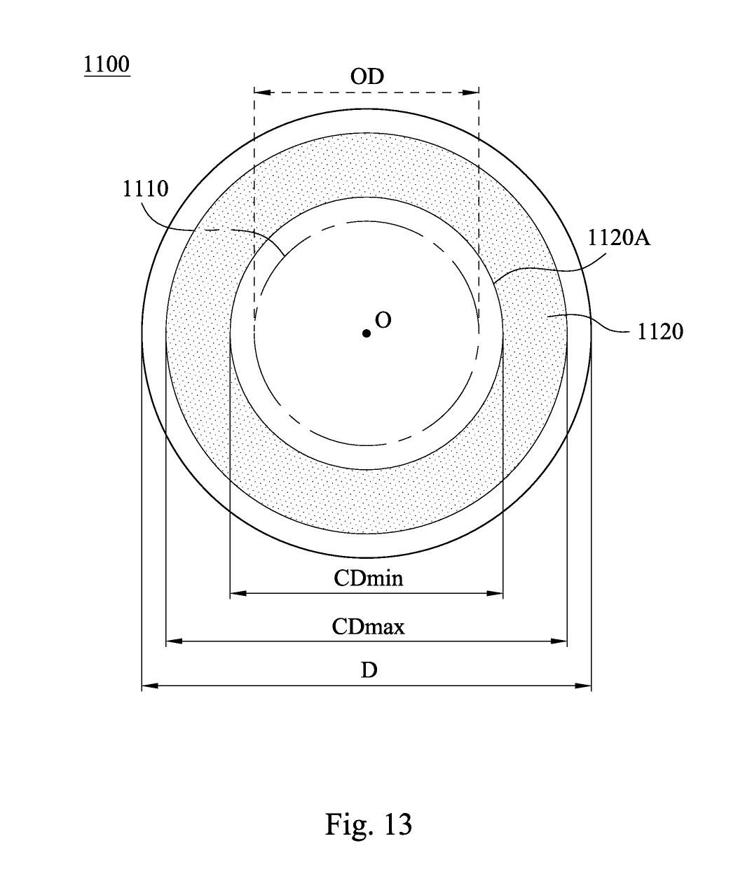

FIG. 13 is a schematic view of a contact lens 1100 according to the 11th example of the present disclosure. In FIG. 13, the contact lens 1100 includes an optical region 1110 and a light-blocking annular region 1120. The light-blocking annular region 1120 surrounds a central point O of the contact lens 1100, and an inner edge 1120A of the light-blocking annular region 1120 is formed in a circle. In the 11th example, the optical region 1110 is the region circled by the dashed line, and the optical region 1110 and the light-blocking annular region 1120 are staggered.

The values of CDmin, CDmax, OD, D, CDmin/D, CDmax/D, CDmax-CDmin, CDmin/OD and BC of the 11th example are listed in Table 21. The definitions of these parameters shown in Table 21 are the same as those stated in the 1st Example, so an explanation in this regard will not be provided to again.

TABLE 21

| | | | |
|---|---|---|---|
| CDmin (mm) | 8.50 | CDmax/D | 0.89 |
| CDmax (mm) | 12.50 | CDmax-CDmin | 4.00 |
| OD (mm) | 7.00 | CDmin/OD | 1.21 |
| D (mm) | 14.00 | BC (mm) | 11.00 |
| CDmin/D | 0.61 | | |

The contact lens 1100 is made of silicone hydrogel, and a composition for manufacturing the silicone hydrogel includes the following monomers: 2-hydroxyethyl methacrylate, N,N-dimethyl acrylamide and 3-(3-methacryloxy-2-hydroxypropoxy)propylbis(trimethylsiloxy)methylsilan. The contact lens 1100 is a multifocal contact lens, and the contact lens 1100 includes a cycloplegic agent.

The light-blocking annular region 1120 includes a photochromic material. In the 11th example, the photochromic material includes 1,3-dihydro-1,3,3-trimethylspiro[2H-indole-2,3′-[3H]naphth[2,1-b][1,4]oxazine] and (E)-benzylidene(diphenylmethylene)succinic anhydride. An average transmittance of visible light of the light-blocking annular region 1120 is Tavg. The light-blocking annular region 1120 is irradiated with a light in a wavelength range of 200 nm to 400 nm for 10 seconds. The values of Tavg before and after irradiating with the light in the wavelength range of 200 nm to 400 nm are listed in Table 22.

TABLE 22

| | |
|---|---|
| (before) Tavg (%) | 90 |
| (after) Tavg (%) | 5 |

### 12th Example

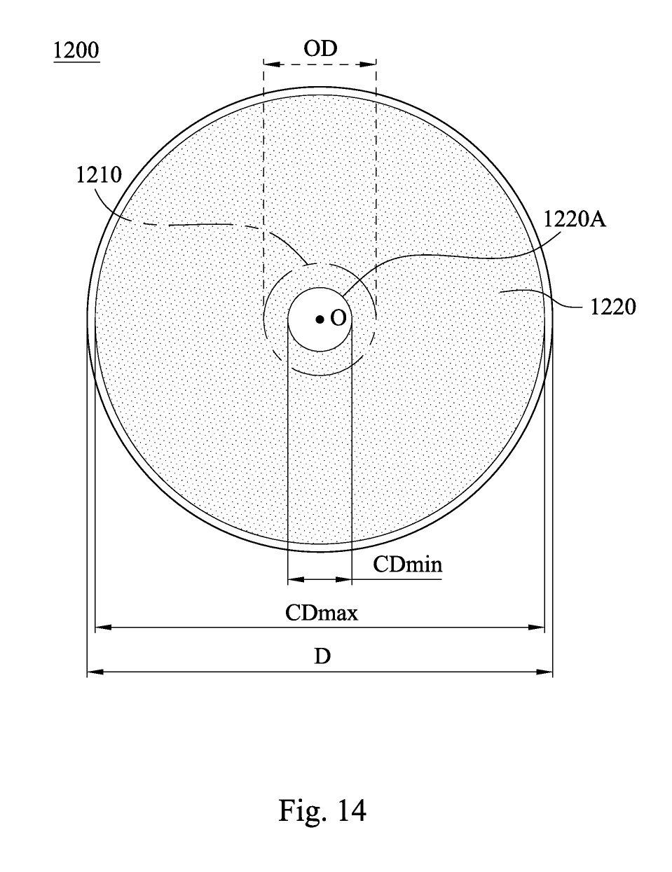

FIG. 14 is a schematic view of a contact lens 1200 according to the 12th example of the present disclosure. In FIG. 14, the contact lens 1200 includes an optical region 1210 and a light-blocking annular region 1220. The light-blocking annular region 1220 surrounds a central point O of the contact lens 1200, and an inner edge 1220A of the light-blocking annular region 1220 is formed in a circle. In the 12th example, the optical region 1210 is the region circled by the dashed line, and the optical region 1210 and the light-blocking annular region 1220 are partially overlapped.

The values of CDmin, CDmax, OD, D, CDmin/D, CDmax/D, CDmax-CDmin, CDmin/OD and BC of the 12th example are listed in Table 23. The definitions of these parameters shown in Table 23 are the same as those stated in the 1st Example, so an explanation in this regard will not be provided again.

TABLE 23

| | | | |
|---|---|---|---|
| CDmin (mm) | 2.00 | CDmax/D | 0.97 |
| CDmax (mm) | 14.00 | CDmax-CDmin | 12.00 |
| OD (mm) | 3.50 | CDmin/OD | 0.57 |
| D (mm) | 14.50 | BC (mm) | 13.50 |
| CDmin/D | 0.14 | | |

The contact lens 1200 is made of silicone hydrogel, and a composition for manufacturing the silicone hydrogel includes the following monomers: 2-hydroxyethyl methacrylate, glycerol monomethacrylate, N-vinyl-2-pyrrolidinone, N, N-dimethyl acrylamide, 3-methacryloyloxypropyltris(trimethylsilyloxy) silane, 3-(3-methacryloxy-2-hydroxypropoxy)propylbis(trimethylsiloxy)methylsilan, (3-acryloxy-2-hydroxypropoxypropyl) terminated polydimethylsiloxane and methyl methacrylate. The contact lens 1200 is a multifocal contact lens.

The light-blocking annular region 1220 includes a photochromic material. In the 12th example, the photochromic material includes 1′,3′-dihydro-1′,3′,3′-trimethyl-6-nitrospiro[2H-1-benzopyran-2,2′-(2H)-indole], hydroxy-4-methyl-2H-naphtho(1,2-B)pyran-2-one and 9,10-dihydrophenanthrene. An average transmittance of visible light of the light-blocking annular region 1220 is Tavg. The light-blocking annular region 1220 is irradiated with a light in a wavelength range of 200 nm to 400 nm for 10 seconds. The values of Tavg before and after irradiating with the light in the wavelength range of 200 nm to 400 nm are listed in Table 24.

TABLE 24

| | |
|---|---|
| (before) Tavg (%) | 45 |
| (after) Tavg (%) | 3 |

### 13th Example

FIG. 15 is a schematic view of a contact lens 1300 according to the 13th example of the present disclosure. In FIG. 15, the contact lens 1300 includes an optical region 1310 and a light-blocking annular region 1320. The light-blocking annular region 1320 surrounds a central point O of the contact lens 1300, and an inner edge 1320A of the light-blocking annular region 1320 is formed in a circle. In the 13th example, the optical region 1310 is the region circled by the dashed line, and the optical region 1310 and the light-blocking annular region 1320 are staggered. In the 13th example, the light-blocking annular region 1320 only includes a light-blocking part (its reference numeral is omitted), and the light-blocking part includes a plurality of sub light-blocking parts 1320a. The sub light-blocking parts 1320a are arranged in a radial pattern, and the average transmittances of visible light of the sub light-blocking parts can be gradually changed. That is, different sub light-blocking parts can have different color shades along the clockwise direction or along the counterclockwise direction. In the 13th example, the color shades of the sub light-blocking parts starting from the twelve o'clock position are arranged to change from light to dark, dark to light, light to dark and then dark to light. In other example, the color shades of the sub light-blocking parts are arranged to change in a different way according to practical demands.

According to the contact lens of the present disclosure, the optical region is a region corresponding to the range of the base curve of the contact lens, i.e., the region having refractive power.

According to the contact lens of the present disclosure, the parameters are not limited to define the front surface or the back surface of the contact lens. That is, the parameters can be designed for the front surface and/or the back surface.

According to the contact lens of the present disclosure, the light-blocking annular region can be formed by transfer printing, printing or coating the light-blocking material on the contact lens. Alternatively, the light-blocking material can be mixed in the composition for manufacturing the light-blocking annular region, so that the light-blocking material is dispersed in the entire of the light-blocking annular region to feature the contact lens with the light-blocking effect. Moreover, the concentration and the color of the light-blocking material can be chosen and adjusted to feature the light-blocking annular region with different degree of transparency for obtaining the desired average transmittance of visible light. The light-blocking material can be the color materials which can be used in medical devices approved by Taiwan Food and Drug Administration (TFDA), U.S. Food and Drug Administration (USFDA) or any of the national food and drug administrations.

According to the contact lens of the present disclosure, the photochromic material can be changed into the color with lower transmittance of visible light by irradiating light (such as blue light or UV), which can effectively reduce or block the visible light passing therethrough. When the irradiating light is disappear or removed, the transmittance of visible light of the photochromic material can be recovered. That is, the color change of the photochromic material is reversible.

According to the contact lens of the present disclosure, the cycloplegic agent can include but is not limited to atropine ((3-endo)-8-methyl-8-azabicyclo[3.2.1]oct-3-yl tropate), tropicamide (N-ethyl-3-hydroxy-2-phenyl-N-(4-pyridinylmethyl)propanamide), cyclopentolate (2-(dimethylamino)ethyl (1-hydroxycyclopentyl)(phenyl)acetate), homatropine ((3-endo)-8-methyl-8-azabicyclo[3.2.1]oct-3-yl hydroxy(phenyl)acetate), scopolamine ((1R,2R,4S,5S,7S)-9-methyl-3-oxa-9-azatricyclo[3.3.1.0^{2,4}]non-7-yl(2S)-3-hydroxy-2-phenylpropanoate), eucatropine (1,2,2,6-tetramethyl-4-piperidinyl hydroxy(phenyl)acetate) or the salt thereof. The cycloplegic agent, also known as a mydriatic agent, belongs to a parasympathetic blocker, i.e., a non-selective m-type muscarinic receptor blocker, which can control the paralysis and relaxation of the ciliary muscle of pupils by blocking the muscarinic receptor so as to enlarge the pupil.

It will be apparent to those skilled in the art that various modifications and variations can be made to the structure of the present disclosure without departing from the scope or spirit of the disclosure. In view of the foregoing, it to is intended that the present disclosure cover modifications and variations of this disclosure provided they fall within the scope of the following claims.

What is claimed is:

1. A contact lens, comprising:
an optical region; and
at least one light-blocking annular region surrounding a central point of the contact lens;
wherein an average transmittance of visible light of the at least one light-blocking annular region is Tavg, a minimum diameter of the at least one light-blocking annular region is CDmin, a maximum diameter of the contact lens is D, and the following conditions are satisfied:
0%<Tavg≦60%; and
0.01≦CDmin/D≦0.45.

2. The contact lens of claim 1, wherein the at least one light-blocking annular region comprises at least two light-blocking parts, and average transmittances of visible light of the light-blocking parts are gradually changed.

3. The contact lens of claim 1, wherein a maximum diameter of the at least one light-blocking annular region is CDmax, the minimum diameter of the at least one light-blocking annular region is CDmin, and the following condition is satisfied:
0.5 mm≦CDmax−CDmin≦13.0 mm.

4. The contact lens of claim 1, wherein the minimum diameter of the at least one light-blocking annular region is CDmin, a maximum diameter of the optical region is OD, and the following condition is satisfied:
0.05≦CDmin/OD≦2.0.

5. The contact lens of claim 1, wherein an inner edge of the at least one light-blocking annular region is formed in a circle or a polygon with at least four sides.

6. The contact lens of claim 1, wherein the contact lens comprises a photochromic material selected from the group consisting of spiropyrans, spirooxazines, benzo and naphthopyrans, fulgides, diarylethenes and a combination thereof.

7. The contact lens of claim 1, wherein the contact lens is made of silicone hydrogel, and a composition for manufacturing the silicone hydrogel comprises at least three kinds of monomers, and the monomers are selected from the group consisting of 2-hydroxyethyl methacrylate, methacrylic acid, glycerol monomethacrylate, N-vinyl-2-pyrrolidinone, N,N-dimethyl acrylamide, 3-methacryloyloxypropyltris(trimethylsilyloxy) silane, 3-(3-methacryloxy-2-hydroxypropoxy)propylbis(trimethylsiloxy)methylsilane, (3-acryloxy-2-hydroxypropoxypropyl) terminated polydimethylsiloxane and methyl methacrylate.

8. The contact lens of claim 1, wherein the contact lens is made of hydrogel, and a composition for manufacturing the hydrogel comprises at least two kinds of monomers, and the monomers are selected from the group consisting of 2-hydroxyethyl methacrylate, methacrylic acid, glycerol monomethacrylate, N-vinyl-2-pyrrolidinone, N,N-dimethyl acrylamide and methyl methacrylate.

9. The contact lens of claim 1, wherein the contact lens is a multifocal contact lens.

10. A contact lens product, comprising:
a contact lens, comprising:
an optical region; and
at least one light-blocking annular region surrounding a central point of the contact lens; and
a soaking solution, wherein the contact lens is immersed in the soaking solution;
wherein at least one of the contact lens and the soaking solution comprises a cycloplegic agent, an average transmittance of visible light of the at least one light-blocking annular region is Tavg, a minimum diameter of the at least one light-blocking annular region is CDmin, a maximum diameter of the contact lens is D, and the following conditions are satisfied:
0%<Tavg≦60%; and
0.01≦CDmin/D≦0.80.

11. The contact lens product of claim 10, wherein the soaking solution comprises the cycloplegic agent.

12. The contact lens product of claim 10, wherein the contact lens comprises the cycloplegic agent.

13. The contact lens product of claim 10, wherein the contact lens comprises a photochromic material selected from the group consisting of spiropyrans, spirooxazines, benzo and naphthopyrans, fulgides, diarylethenes and a combination thereof.

14. The contact lens product of claim 10, wherein the contact lens is a multifocal contact lens.

15. A contact lens, comprising:
an optical region; and
at least one light-blocking annular region surrounding a central point of the contact lens;
wherein at least one of the optical region and the at least one light-blocking annular region comprises a photochromic material, an average transmittance of visible light of the at least one light-blocking annular region is Tavg, a minimum diameter of the at least one light-blocking annular region is CDmin, a maximum diameter of the contact lens is D, and the following conditions are satisfied:
0%<Tavg≦60%; and
0.01≦CDmin/D≦0.80.

16. The contact lens of claim 15, wherein a maximum diameter of the at least one light-blocking annular region is CDmax, the maximum diameter of the contact lens is D, and the following condition is satisfied:
0.5≦CDmax/D≦1.0.

17. The contact lens of claim 15, wherein the optical region comprises the photochromic material selected from the group consisting of spiropyrans, spirooxazines, benzo and naphthopyrans, fulgides, diarylethenes and a combination thereof.

18. The contact lens of claim 15, wherein the at least one light-blocking annular region comprises the photochromic material selected from the group consisting of spiropyrans, spirooxazines, benzo and naphthopyrans, fulgides, diarylethenes and a combination thereof.

19. The contact lens of claim 15, wherein the contact lens is a multifocal contact lens.

20. A contact lens, comprising:
an optical region; and
at least one light-blocking annular region surrounding a central point of the contact lens;
wherein an average transmittance of visible light of the at least one light-blocking annular region is Tavg, a minimum diameter of the at least one light-blocking annular region is CDmin, a maximum diameter of the contact lens is D, a base curve of the contact lens is BC, and the following conditions are satisfied:
0%<Tavg≦60%;
0.01≦CDmin/D≦0.80; and
9.00 mm≦BC.
